(19) United States
(12) Patent Application Publication
(10) Pub. No.: US 2025/0236893 A1
(43) Pub. Date: Jul. 24, 2025

(54) ENHANCING THE SEQUESTRATION OF CARBON DIOXIDE IN DOWNHOLE CARBONATE-BASED RESERVOIRS VIA INJECTING UREASE ENZYME

(71) Applicant: SAUDI ARABIAN OIL COMPANY, Dhahran (SA)

(72) Inventors: Abeer A. Alarawi, Al Khobar (SA); Manar M. AlAhmari, Dhahran (SA)

(73) Assignee: SAUDI ARABIAN OIL COMPANY, Dhahran (SA)

(21) Appl. No.: 18/419,342

(22) Filed: Jan. 22, 2024

(51) Int. Cl. C12P 3/00

(52) U.S. Cl. CPC C12P 3/00

(57) ABSTRACT

A method of biomineralizing carbon dioxide (CO₂) in a formation includes injecting a CO₂-containing fluid and an Enzyme Induced Carbonate Precipitation (EICP) solution in the formation to form a mineralization mixture and forming a carbonate precipitate in the formation from the mineralization mixture. The EICP solution may include urea, one or more polysaccharides, a casein protein, a protease, an ionic compound, and a urease.

### BACKGROUND

It is now widely accepted that anthropogenic carbon dioxide (CO₂) emissions tend to throw the natural carbon cycle out of balance, where CO₂ resulting from all natural and anthropogenic carbon sources are taken up by CO₂ sinks. As a consequence, a significant fraction of the CO₂ that is emitted on an annual basis tends to reside in the atmosphere. Despite the emerging widespread and very significant efforts to reduce CO₂ emissions worldwide as well as efforts to transition from fossil fuels to clean and renewable energies, petroleum production will likely continue in the foreseeable future to meet the world's growing energy demand, leading to continuing CO₂ emissions. Therefore, there has been interest in CO₂ storage in order to remove the CO₂ from the atmosphere.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method of biomineralizing carbon dioxide (CO₂) in a formation that includes injecting a CO₂-containing fluid and an Enzyme Induced Carbonate Precipitation (EICP) solution in the formation to form a mineralization mixture, and forming a carbonate precipitate in the formation from the mineralization mixture. The EICP solution may include urea, one or more polysaccharides, a casein protein, a protease, an ionic compound, and a urease.

Other aspects and advantages of the claimed subject matter will be apparent from the following description and the appended claims.

### DETAILED DESCRIPTION

Carbon dioxide (CO₂) is a naturally occurring compound that is present in Earth's atmosphere. The CO₂ in the atmosphere may be derived from natural sources, such as respiration, or from human activities, such as the combustion of fossil fuels. The environmental effects of CO₂ in the atmosphere are of particular concern because CO₂ is a “greenhouse gas”. A greenhouse gas can absorb light and radiate heat instead of reflecting it, elevating the temperature of the gas.

In efforts to slow the rate of global warming, carbon capture and storage (CCS) has emerged as a possible solution for reducing CO₂ in the atmosphere. In a typical CCS process, atmospheric CO₂ is captured, compressed, and transported with the eventual goal of long-term storage in underground geological formations. Since the industrial revolution, an enormous increase in the atmospheric concentration of CO₂ and other Green-House Gases (GHGs) has been detected. Moreover, the average global surface temperature has increased due to the increased concentration of GHGs in the atmosphere, which causes an annual increase in average temperature over most of Australia of 0.4 to 2.0° C. from 1990 to 2030 and may increase by about 1 to 6° C. by 2070.

Several solutions and measures have been studied to reduce the amount of CO₂ emissions and develop methods to mitigate global warming. The controlled solutions that may prevent or delay excessive anthropogenic CO₂ from reaching the atmosphere may include using less carbon intensive fuels, improving energy efficiency, and pursuing carbon capture sequestration (CCS) through different means.

In general, CCS is defined as an industrial process to store CO₂ deep under the earth surface, deep into the ocean, or both. Hence, CCS avoids the CO₂ greenhouse impact of reducing anthropogenic CO₂ emission. The current applied sequestration techniques essentially rely on knowledge and experience gained from oil and gas industry, coal-bed methane, and underground natural gas storage. In fact, applying these techniques for CO₂ sequestration have several advantages including improving CO₂ sequestration in geologic formations, reduce costs, increase capacity, enhance safety, or increase the beneficial uses of CO₂ injection. Such enhanced methods may include improved mineral trapping with utilizing catalysts or other chemical additives, sequestration in composite formations that are multilayered geological formations of imperfect rocks, which result in greater dispersion of a CO₂ plume, reconstruction of depleted oil reservoirs through enhanced oil recovery (EOR), enhanced production of methane hydrates by injecting CO₂ into methane hydrate formations while simultaneously storing CO₂, or combinations thereof.

Carbon dioxide (CO₂) can be sequestered in geologic formations by four main mechanisms that include structural stratigraphic trapping, capillary residual trapping, solubility trapping, and mineral trapping. The efficiency of these CO₂ sequestration methods into formations depends on a storage capacity of the formation, reservoir stability and potential risk of leakage. For example, CO₂ may be dissolved in a first aqueous fluid and injected in a formation in which mineralization may occur.

Mineral trapping includes chemical reactions between gas and/or liquid phases. In general, kinetic aspects of the mechanism(s) involved in mineral trapping are not as well understood. For example, pressure and temperature play only a small role in the mineral trapping process. Additionally, rapid mineral dissolution in carbonate-bearing formations can have considerable environmental implications due to the creation of pathways for fluid flow in carbonate rock seals and well cements that could facilitate leakage of supercritical CO₂ and brine. This kind of dissolution should be carefully monitored in order to prevent the deterioration of caprock integrity. Thus, the factors to be considered in the geological storage of CO₂ are sweep efficiency, preferential flow, leakage rates, CO₂ dissolution kinetics, mineral trapping kinetics, microbial interactions with CO₂, and the influence of stress changes on caprock and formation integrity.

Embodiments herein are related to compositions, systems, and methods for enhancing CO₂ sequestration in a downhole carbonate-based formation. The carbonate-based formations of one or more embodiments may have a temperature in a range from about 150 to about 325° F. For example, the carbonate-based formation may have a temperature in a range from about 200 to about 300° F. Compositions, systems, and methods in accordance with one or more embodiments may use enzyme induced calcium carbonate precipitation solution (EICP) to sequester CO₂ in a formation as mineral carbonates. The use of EICP compositions in a system and method of one or more embodiments can reduce the migration of an external source of CO₂ injected into a formation by converting it into a metal carbonate including, but not limited to, calcium carbonate (CaCO₃) utilizing an EICP system. The treatment compositions, treatment systems, and methods described herein may be configured to sequester CO₂ from a CO₂-containing fluid in a formation in the presence of an EICP composition. Treatment compositions and methods herein may include simple, economically and environmentally friendly biological treatment agents.

The compositions, systems, and methods may be capable of promoting an EICP reaction to enhance a rate of mineralization of CO₂ in carbonate formations, increase a CO₂ sequestration efficiency, or both. The rate of mineralization of CO₂ in carbonate formations according to a treatment method of one or more embodiments is enhanced compared to a rate of mineralization without an EICP composition and a treatment method of one or more embodiments. In one or more embodiments, one or more formation parameters, such as minerals present in the formation, a CO₂ phase, formation pressure and formation temperature, or combinations thereof, further enhances the rate of CO₂ mineralization along with the EICP reaction.

### EICP Solution Composition

One or more embodiments of the present disclosure relate to an aqueous treatment composition including an EICP solution that is capable of hydrolyzing urea with a urease enzyme in an aqueous solution and forming carbonic acid and ammonia. The aqueous EICP solution is a homogenous mixture of different additives (i.e., a plurality of components). The plurality of components may include urea, one or more polysaccharides, casein protein, protease, an ionic compound, and a urease.

In one or more embodiments, the aqueous EICP solution includes urea. Urea is an organic compound of the chemical formula CO(NH₂)₂. Urea is a colorless, odorless, water soluble substance with low toxicity (e.g, LD₅₀=12 g/kg (grams of substance per kilogram of body weight) for mouse, Agrium Material Safety Data Sheet (MSDS)). Any suitable source of urea may be used. In one or more embodiments, the urea is present in the EICP solution at concentrations in a range of from about 0.45 M (moles per liter of aqueous solution) to about 1.55 M urea, such as from about 0.6 M to about 1.4 M, or from about 0.7 M to about 1.3 M, or from about 0.8 M to about 1.2 M. Urea may be present in the EICP solution at a concentration in a range having a lower limit of any one of 0.45 M, 0.5, 0.6, 0.7, 0.8, 0.9, and 1.0 M and an upper limit of any one of 0.7, 0.8, 0.9, 1.0, 1.2, 1.5, and 1.55 M, where any lower limit can be paired with any mathematically compatible upper limit.

In one or more embodiments, the EICP solution includes the enzyme urease. The urease of the solution may be synthetically produced or obtained by extraction from any suitable source, including but not limited to, bacteria, plants, invertebrates, and fungi. In one or more embodiments, a plant derived urease extract may be used. In one or more particular embodiments, the urease included in the EICP solution is a urease enzyme isolated and recovered from a Jack Bean plant. In one or more embodiments, the EICP solution includes urease at concentrations in a range of from about 0.95 g/L (gram per Liter of aqueous solution to about 4.1 g/L, from about 1 g/L to about 4 g/L, or from about 1.5 g/L to about 3.5 g/L, or from about 2 g/L to about 3 g/L. Urease may be present in the EICP solution at a concentration in a range having a lower limit of any one of 0.95 g/L, 1.0, 1.2, 1.5, 1.75, 1.9, 2.0, 2.5, and 3.0 g/L and an upper limit of any one of 2.5, 2.95, 3.0, 3.2, 3.5, 3.8, 3.9, 4.0, and 4.1 g/L, where any lower limit can be paired with any mathematically compatible upper limit.

In one or more embodiments, the polysaccharide of the aqueous EICP solution may comprise xanthan gum. In one or more embodiments, the polysaccharide may comprise a galactomannan polysaccharide, such as guar gum, at concentrations in a range of from about 0.45 M to about 1.05 M. The polysaccharide may be present in the EICP solution at a concentration in a range having a lower limit of any one of 0.45 M, 0.5, 0.6, 0.7, 0.75, 0.8, 0.85, 0.9, and 0.95 M and an upper limit of any one of 0.7, 0.75, 0.8, 0.85, 0.9, 0.95, 1.0, and 1.05 M, where any lower limit can be paired with any mathematically compatible upper limit.

In one or more embodiments, the casein protein of the aqueous EICP solution includes a micellar casein protein. The casein protein may be a micellar casein protein. The solution may include casein protein at concentrations in a range of from about 1.95 g/L (gram per liter of aqueous solution) to about 4.05 g/L. The casein protein may be present in the EICP solution at a concentration in a range having a lower limit of any one of 1.95 g/L, 2.0, 2.2, 2.5, 2.75, 2.9, 3.0, and 3.5 g/L and an upper limit of any one of 2.5, 2.95, 3.0, 3.2, 3.5, 3.8, 3.9, 4.0, and 4.05 g/L, where any lower limit can be paired with any mathematically compatible upper limit.

In one or more embodiments, the aqueous EICP solution may include one or more ionic compounds, such as sodium chloride. One or more ionic compounds may be present in the EICP solution at concentrations in a range of from about 0.35 g/L (gram per liter of aqueous solution) to about 0.65 g/L. The one or more ionic compounds may be present in the EICP solution at a concentration in a range having a lower limit of any one of 0.35 g/L, 0.38, 0.40, 0.42, 0.45, and 0.5 g/L and an upper limit of any one of 0.55, 0.58, 0.60, 0.62, and 0.65 g/L, where any lower limit can be paired with any mathematically compatible upper limit.

The EICP solution may also include a sugar, one or more sugar derivatives, or mixtures thereof. For example, the solution may include sucrose, one or more sucrose derivatives, or mixtures thereof. The sugar, one or more sugar derivatives, or mixtures thereof at concentrations in a range of from about 0.35 g/L (gram per liter of aqueous solution) to about 0.65 g/L. The sugar, one or more sugar derivatives, or mixtures thereof may be present in the EICP solution at a concentration in a range having a lower limit of any one of 0.35 g/L, 0.38, 0.40, 0.42, 0.45, and 0.5 g/L and an upper limit of any one of 0.55, 0.58, 0.60, 0.62, and 0.65 g/L, where any lower limit can be paired with any mathematically compatible upper limit.

Furthermore, the EICP solution may also include a protease, for example, a protease that may be obtained from Aspergillus niger or Aspergillus oryzae. In some embodiments, the protease includes a protease obtained from Aspergillus niger, Aspergillus oryzae, or combinations thereof. For example, the protease may include Aminogen® (commercially available from Innophos), which is a mixture of proteases obtained from Aspergillus niger and Aspergillus oryzae. Aminogen® is a protein digesting enzyme (protease) that catalyzes the breakdown of proteins into smaller polypeptides or amino acids by breaking the peptide bonds between the amino acids. The EICP solution may include a protease at concentrations in a range of from about 0.35 g/L (gram per liter of aqueous solution) to about 0.85 g/L. The protease may be present in the EICP solution at a concentration in a range having a lower limit of any one of 0.35 g/L, 0.38, 0.40, 0.42, 0.45, 0.5, 0.55, and 0.6 g/L and an upper limit of any one of 0.55, 0.58, 0.60, 0.62, 0.65, 0.70, 0.75, 0.80 and 0.85 g/L, where any lower limit can be paired with any mathematically compatible upper limit.

In some embodiments, the EICP solution is introduced into a formation as one solution. In some embodiments, the EICP solution is introduced into the formation as two or more solutions. For example, a first solution and a second solution may be introduced to the formation such that the EICP solution is a combination of a first solution and a second solution. In such embodiments, the first solution includes urea, one or more polysaccharides, casein protein, protease, and an ionic compound. The second solution may include a urease.

The EICP solutions of traditional systems can only be applied at temperatures up to 70° C. the enzyme urease denatures and becomes in active at temperatures of 70° C. and above. In contrast, the EICP composition according to one or more embodiments herein is capable of withstanding high temperatures, such as formation temperatures up to 130° C. In particular embodiments, the addition of one or more polysaccharides, casein protein, protease, and an ionic compound stabilizes the urease enzyme at a temperatures of 70° C. and above (e.g., to an upper limit of 130° C.) and allows for the generation of nucleation points for carbonate precipitation.

### Treatment Composition

In another aspect, embodiments herein relate to a treatment composition to sequester CO₂. The treatment composition may include an EICP solution and an amount of CO₂-containing fluid. The treatment composition may be a mineralization mixture configured to produce carbonate minerals. The EICP solution may be as described above. The CO₂-containing fluid may include a source of CO₂. The source of CO₂ may be CO₂ that has been captured and compressed. In some embodiments, the source of CO₂ includes supercritical CO₂. In some embodiments, the CO₂-containing fluid includes an aqueous fluid. In particular embodiments, the CO₂-containing fluid may include a carbonate precipitating agent. In such embodiments, the carbonate precipitating agent may be dissolved in an aqueous fluid. The carbonate precipitating agent may include a metal cation source. The metal cation source may be a calcium ion source, such as calcium chloride dihydrate. A source of the carbonate precipitating agent dissolved in the produced water may be a formation rock of a reservoir, such that the produced water carries or dissolves the carbonate precipitating agent from the formation to a surface of the reservoir. In some embodiments, the carbonate precipitating agent includes a metal cation. The metal cation may include, but is not limited to, one or more alkali metal cations, alkaline earth metal cations, transition metal cations, or combinations thereof.

In one or more embodiments, the metal cation may include one or more metal-containing minerals, such as calcium containing minerals, magnesium containing minerals, iron containing minerals, or combinations thereof. Non-limiting examples of metal-containing minerals include fosterite, plagioclase, or combinations thereof. The metal cation may be obtained from a source that can include a downhole formation rock of the formation. The formation rock may include ultra-mafic rock, mafic rock, basaltic rock, or combinations thereof.

In one or more embodiments, the CO₂-containing fluid includes a water-based fluid. The water-based fluid may be distilled water, brine, deionized water, tap water, fresh water from surface or subsurface sources, formation water produced from the structural low, formation water produced from a different geologic formation, production water, frac or flowback water, natural and synthetic brines, residual brine from desalination processing, a regional water source, such as fresh water, brackish water, natural and synthetic sea water, potable water, non-potable water, ground water, seawater, other waters, and combinations thereof, that are suitable for use in a wellbore environment. In one or more embodiments, the water used may naturally contain contaminants, such as salts, ions, minerals, organics, and combinations thereof, as long as the contaminants do not interfere with the precipitation of CO₂ from the CO₂-containing water, a thermogenic reaction, or both. In one or more embodiments, the water-based fluid includes additives such as viscosifiers, polymers, surfactants, and combinations thereof. In one or more particular embodiments, the CO₂-containing fluid includes an amount of water that is abundant in calcium ions, such as seawater, produced water, or both.

The water-based fluids of one or more embodiments may include other additives provided the additives do not interfere with the precipitation of CO₂ from the CO₂-containing fluid and EICP processes. Such additives may include, for instance, one or more wetting agents, corrosion inhibitors, biocides, surfactants, dispersants, interfacial tension reducers, mutual solvents, and thinning agents. The identities and use of the aforementioned additives are not particularly limited. One of ordinary skill in the art will, with the benefit of this disclosure, appreciate that the inclusion of a particular additive will depend upon the stage of reservoir operations, desired application, and properties of a given wellbore fluid.

In one or more embodiments, the solubility of CO₂ is salinity dependent. As salinity of the aqueous fluid decreases, the solubility of CO₂ in the aqueous fluid increases. Generally, CO₂ solubility in brine decreases as salinity increases due to the so-called “salting out” effect. Comparisons between different salinities indicate that CO₂ solubility decreases nearly 50% when the salinity increases from about 0 M (Molar) to about 4 M aqueous NaCl solutions. In one or more embodiments, more CO₂ is dissolved in a water-based fluid as described above with a lower salinity than a water-based fluid with high salinity. A lower amount of a water-based fluid may be needed when water of low salinity is used for the same amount of CO₂ available under the same temperature and pressure conditions when compared to a high salinity water. In addition, when more CO₂ is dissolved in water, the pH of the water decreases. The CO₂ may be dissolved in a sufficient amount to maximize an efficiency of the mineralization process via EICP biomineralization. In one or more embodiments, the amount of CO₂ dissolved in the first aqueous fluid may be dependent upon a temperature and/or pressure of the formation.

A ratio of the EICP solution to the CO₂-containing fluid may be a ratio in a range from about 1:4.2 to about 1:3.8 to affect CO₂ sequestration and carbonate formation. In one or more particular embodiments, the ratio of the EICP solution to the CO₂-containing fluid is present in the treatment composition at a ratio of about 1:4 to affect CO₂ sequestration and carbonate formation. The composition and ratio of the EICP solution to the CO₂-containing fluid may promote faster biomineralization of CO₂ into carbonate minerals as compared to other CO₂ mineralization and sequestration methods.

### System for Treating a Formation

In another aspect, embodiments herein relate to a system for treating a formation that is configured to sequester CO₂ in a formation. The system of one or more embodiments may be configured to facilitate the biomineralization of an external source of CO₂ (e.g., CO₂ present in a CO₂-containing fluid), such that the external source of CO₂ is sequestered in the formation as a solid carbonate precipitate. The system may be configured to sequester CO₂ with an EICP solution as described above. In one or more embodiments, the system includes an injection system, one or more fluid transport lines, one or more mixing units configured to prepare one or more of a CO₂-containing fluid, a first solution, a second solution, an EICP solution, or combinations thereof.

The system of one or more embodiments may include one or more mixing tanks in fluid communication with an injection system. The one or more mixing tanks may include one or more fluid feed lines and additional mixing units such that the mixing tanks may be configured to prepare a first solution, a second solution, an EICP solution, a CO₂-containing fluid, dissolving a precipitating agent in one or more fluids of the treatment system, or combinations thereof. In one or more particular embodiments, the precipitating agent may be dissolved in the CO₂-containing fluid, a first solution capable of forming an EICP solution, an EICP solution, or combinations thereof.

The injection system in fluid communication with an injection well of the formation. The system may transport a CO₂-containing fluid, and EICP solution or a first salt solution, and a second salt solution to a downhole location in the formation. The CO₂-containing fluid, the EICP solution, the first solution, and the second solution may be as described above. In one or more embodiments, the system may be configured to solubilize CO₂ in a sufficient amount of an aqueous fluid such that the gas is completely dissolved and maintains the form of a CO₂-containing fluid at the depth of the release into the target subterranean formation.

The system may include multiple injection lines in fluid communication with the injection well of the formation. The injection lines may be configured to separately inject one or more fluids into the injection well of the formation such that a treatment composition or a mineralization mixture (e.g., a mixture of the EICP solution, an amount of CO₂-containing fluid, and a precipitating agent) is formed in a downhole location. In such embodiments, the system may include two or more, three or more, or four or more injection lines in fluid communication with an injection well of a formation. The injection lines of the system may be capable of separately injecting a first solution and a second solution to the formation such that the EICP solution is formed downhole. In such instances, the CO₂-containing fluid may be injected separately by an additional injection line in fluid communication with the formation. The multiple injection lines of the system may be capable of separately injecting a CO₂-containing fluid and an EICP solution into the formation.

The system may be configured to separate the injection of the first solution and the second solution, where the second solution is capable of an EICP reaction with the first solution upon contact, under injection conditions and under conditions in the formation to initiate the EICP process. For example, the injection system may include a first solution transport line configured to inject the first solution composition. The second solution transport line may be inserted into the injection well of a formation. The injection system may include a second solution transport line configured to inject the second solution composition. The second solution transport line may be inserted into the injection well of a formation. The injection system may include a CO₂-containing fluid transport line configured to inject the CO₂-containing fluid composition. The injection system may include an EICP solution transport line configured to inject the CO₂-containing fluid composition. In one or more embodiments, the first salt solution transport line and the CO₂-containing fluid transport line are the same.

### Method for Sequestering CO₂

In another aspect, one or more embodiments relate to a method of treating a carbonate formation. The treatment may include sequestering CO₂ in the carbonate formation from a CO₂-containing fluid with an EICP solution of one or more embodiments. The method of one or more embodiments may include sequestering CO₂ from extraneous CO₂ injected in a CO₂-containing fluid via transformation to a solid carbonate form by biomineralization. A method of one or more embodiments may include a system for treating a formation including sequestering CO₂ via biomineralization.

The method of one or more embodiments includes injecting a CO₂-containing fluid into the formation. The CO₂-containing fluid may be injected into the carbonate formation via a CO₂-containing fluid transport line of a system, which may be as described above. In some embodiments, the method includes injecting captured and compressed CO₂ in the formation. The method may include injecting supercritical CO₂ in the formation. The method of one or more embodiments may include capturing CO₂ via solvation in an aqueous fluid. Capturing and dissolving CO₂ in an aqueous fluid may be performed in a system as described above. The solubilization of CO₂ in the aqueous fluid may be represented by Equation (1), below.

$${CO}_{2} ( g ) + {H}_{2} O ( aq ) ⇌ {H}_{2} {CO}_{3} ( a q ) \qquad Equation ( 1 )$$

As provided above, CO₂ gas may react with water (H₂O) to dissolve and provide aqueous carbonic acid (H₂CO₃). Once dissolved in water in the form of carbonic acid, CO₂ may no longer be buoyant, providing a dense and acidic CO₂-containing fluid. The acidity of the CO₂-containing fluid may be represented by the reversible dissociation of carbonic acid in Equation (3), below.

$${H}_{2} {CO}_{3} ( aq ) ⇌ {HCO}_{3}^{-} ( a q ) + {H}^{+} ( a q ) \qquad Equation ( 2 )$$

As such, free hydrogen of dissolved CO₂ may provide an acidic CO₂-containing fluid with a pH of about 7.0 or less, about 6.0 or less, about 4.0 or less, about 3.0 or less, or about 2.0 or less. As a result, it is expected that when more CO₂ is dissolved in a lower salinity water-based fluid to produce a CO₂-containing fluid with a pH of about 7.0 or less, reactivity with one or more metal cations from a formation and one or more components of the EICP solution may be expedited such that a rate of carbonate precipitate formation is enhanced. In one or more embodiments, the system facilitates a CO₂-containing fluid contacting an EICP solution and a metal cation downhole.

The method may include forming a mineralization mixture downhole. The mineralization mixture may include an EICP solution, a CO₂-containing fluid, and one or more precipitating agents. The method of one or more embodiments may include introducing one or more metal ions as a precipitating agent from the system to the formation. In some embodiments, the precipitating agent may be introduced to the formation along with the CO₂-containing fluid, an EICP solution, a first solution, a second solution, or combinations thereof. The method may include injecting one or more fluids to a downhole formation location to facilitate an EICP reaction and the sequestration of an external source of CO₂ (e.g., CO₂ from the CO₂-containing fluid). In some embodiments, a carbonate precipitate is formed in the formation from the mineralization mixture.

The EICP process of one or more embodiments is an in situ chemical reaction that may be a useful part of a method for effecting sequestration of CO₂ in a formation. EICP according to one or more embodiments herein employs a urease enzyme to catalyze the hydrolysis of urea in an aqueous solution. EICP in the presence of divalent ions generates ammonium ions and a carbonate mineral that precipitates out of the aqueous solution. For example, in the presence of calcium ions, the EICP process results in calcium carbonate (CaCO₃) precipitate as shown in Equation 3:

$${H}_{2} N - CO - {NH}_{2} + 2 {H}_{2} O + {Ca}^{2 +} {⟺}^{urease} 2 {NH}_{{4}^{+}} + {CaCO}_{3} ( s ) . \qquad Equation ( 3 )$$

Mineral carbonate precipitation may include one or more cations present in precipitating agent, the CO₂-containing fluid, the EICP solution, or combinations thereof that may produce one or several phases of carbonate mineral precipitate, including, but not limited to, barite, struvite, calcite, halite, nesquehonite, or combinations thereof. The method for reducing the ionic concentration of a produced water described in one or more embodiments herein takes advantage of the supply of carbonate ions derived from urea hydrolysis and an increase in pH generated by the reaction.

The increase in pH generated by the reaction may be a result of the formation of ammonia in the treatment composition. For example, the production of hydroxide ions from ammonia reaction with water molecules brings about an increase in pH, which in turn leads to the formation of carbonate ions from the produced CO₂. The produced CO₂ may dissolve in water to form carbonic acid and bicarbonate ion via a mechanism as described in Equations (1) and (2) above. EICP may increase the pH level of the solution, which favors bicarbonate ions reaction with free ions in the solution to form carbonates. For instance, urease mediated urea hydrolysis reaction in the presence of calcium ions, precipitates calcium carbonate. For example, EICP may increase the pH of the solution to a range from about 8.0 to about 10.0. EICP may increase the pH of the solution to a range having a lower limit of 8.0, 8.2, 8.5, 8.7, 8.8, and 9.0 and an upper limit of any one of 8.5, 8.7, 8.9, 9.0, 9.2, 9.5, 9.6, 9.8, 9.9, and 10.0, where any lower limit can be paired with any mathematically compatible upper limit.

The EICP process may be triggered by the catalytic action of an enzyme in the hydrolysis of urea. For example, EICP in accordance with one or more embodiments is a method of carbonate precipitation via hydrolysis of urea employing urease enzyme. One or more components of the EICP solution may stabilize the urease enzyme and act as nucleation point for ions, thereby enhancing ionic precipitation from produced water. The method of one or more embodiments utilizes the urease enzyme to catalyze the hydrolysis of urea (ureolysis) in an aqueous solution including metallic ions (e.g., produced water as described above), and promotes the formation of carbonate precipitates (e.g., calcium carbonate precipitates, magnesium carbonate precipitates, among others).

The chemical composition of the EICP solution may be configured to provide nucleation sites that favor carbonate precipitate formation. The increase in the number of nucleation sites allows for the enhanced precipitation of ions. Accordingly, the method of contacting a CO₂-containing fluid with an EICP solution and a precipitating agent may provide for the formation of a treatment mixture that sequesters CO₂ as a carbonate precipitate. In some embodiments, providing the reaction between the one or more metal ions and the CO₂ from the CO₂-containing fluid includes shifting the equilibrium of the reaction. Equation (4), below, shows a non-limiting example of a reaction of calcium carbonate formation.

$${Ca}^{2 +} ( a q ) + {CO}_{3}^{2 -} ( aq ) ⇌ {CaCO}_{3} ( s ) \qquad Equation ( 4 )$$

The method of one or more embodiments may be capable of shifting the equilibrium of a carbonate forming reaction (e.g., the reaction of Equation (4)) to the formation of solid carbonates (e.g., CaCO₃) such that the saturation state of solid carbonate precipitate is exceeded. This shift in equilibrium is achievable only when sufficient ions in the precipitating agent is present, if pH is increased, and if nucleation substrates are present. The increase in pH, CO₃⁻², and HCO³⁻ occurs via EICP reaction, which favors CaCO₃ precipitation. External CO₂ from the CO₂-containing fluid may be sequestered via mineral-trapping, where carbonate ions from the injected CO₂ can be incorporated into a stable solid carbonate mineral (CaCO₃) that is produced via EICP reaction.

In some embodiments, the EICP solution is injected into the formation as a single solution such that the EICP solution is formed prior to injection into the formation (e.g., at a surface location of the formation). In one or more embodiments, the method includes injecting a first solution and a second solution in the formation such that an EICP solution is formed downhole. The first solution and the second solution may be as described above. The first solution and the second solution may be injected separately into the formation such that the EICP solution is formed in the formation upon combination of the first solution and the second solution. The first solution and the second solution may be injected sequentially or simultaneously into the formation such that the EICP forms in the formation from the combination of the first solution and the second solution. In one or more embodiments, the first solution and the second solution may be injected separately via a first solution transport line and a second solution transport line of the system for treating a formation.

In one or more embodiments, the CO₂-containing fluid and the first solution and the second solution are introduced into the subterranean formation sequentially. In one or more embodiments, the first solution and the second solution and the CO₂-containing fluid are introduced into the subterranean formation simultaneously. In one or more embodiments, the CO₂-containing fluid and the EICP solution are introduced into the subterranean formation sequentially. In one or more embodiments, the EICP solution and the CO₂-containing fluid are introduced into the subterranean formation simultaneously. In one or more particular embodiments, the CO₂-containing fluid is injected into the formation prior to the injection of the EICP solution or the first solution and the second solution. In one or more embodiments, the EICP solution or the first solution and the second solution and the CO₂-containing fluid are simultaneously introduced to the formation via separate fluid transport lines as described above such that the solutions are mixed in the formation. In one or more embodiments, the CO₂-containing fluid is injected into the formation prior to the injection of the EICP solution or the first solution and the second solution.

In some embodiments, the presence of the EICP solution downhole in a formation enhances the reactivity of CO₂ from the CO₂-containing fluid with a precipitating agent (e.g., a metal cation). A parameter that may further enhance the reactivity of CO₂-containing fluid with one or more metal cations of a carbonate formation is the acidity of the CO₂-containing fluid. In one or more embodiments, the CO₂-containing fluid reacts with one or more metal cations to generate one or more metal cations. In one or more embodiments, the metal-containing minerals may react with free hydrogens of an acidic solution, such an acidic CO₂-containing fluid to generate one or more metal cations.

In some embodiments, the mixture of the CO₂-containing fluid and the EICP solution in the formation promotes a reaction between CO₂ in the CO₂-containing fluid and one or more metal ions in the mixture. Furthermore, the change in brine/formation water chemistry during ureolysis may also enhance the solubility trapping capacity of the brine through pH changes and this carbonate ion speciation.

The one or more metal cations in a mineralization mixture (e.g., a mixture of a CO₂-containing fluid, a precipitating agent, and an EICP solution) may react with dissolved CO₂ in the form of carbonic acid to form one or more metal carbonates as described in Equation (5).

$${( Ca , Mg , Fe )}^{2 +} ( a q ) + {H}_{2} {CO}_{3} ( a q ) \to ( Ca , Mg , Fe ) {CO}_{3} ( s ) + 2 {H}^{+} ( a q ) \qquad Equation ( 5 )$$

The one or more metal carbonates may be one or more solid metal carbonates. The one or more metal carbonates may include, but are not limited to, calcium carbonate, magnesium carbonate, iron carbonate, aluminum carbonate, or combinations thereof. The degree to which the generated one or more cations form stable carbonate minerals may depend on the identity of the metal ion, a pH of a mineralization mixture, and a temperature of a mineralization mixture. In one or more embodiments, the reaction between the one or more metal ions and the CO₂ from the mineralization mixture precipitating CO₂ from the mineralization mixture in the form of one or more solid metal carbonates at a downhole location of the formation. The solid metal carbonate precipitate formed at a downhole location may settle in the formation, thereby sequestering the precipitated CO₂ in the carbonate formation. The increased rate of one or more solid metal carbonate formation may be an enhanced rate of precipitation of CO₂ from a mineralization mixture including an EICP solution, a CO₂-containing fluid, and a precipitating agent.

In one or more embodiments, the reaction of Equation (5) occurs if the hydrogen ions produced in Equation (5) are consumed by an additional reaction. In one or more embodiments, the additional reaction is hydrogen transfer to a byproduct of the EICP process. For example, as one of ordinary skill may appreciate, the byproducts of the Equation (3) are CO₂, ammonia, and hydroxide. The hydrogen ions produced from Equation (5) may with the CO₂ produced from Equation (3), the hydroxide ions, or both, thereby perpetuating the equilibrium shift of Equation (3) to favor the formation of carbonate of carbonate precipitate. The method of one or more embodiments may lead to a chemical reaction that can be carried out at temperatures in a range from room temperature (e.g., 20-25° C.) to about 130° C.

In some embodiments, the method includes maintaining the formation such that the mineralization mixture achieves a temperature in a range from about 20° C. to about 130° C. In some embodiments, where one or more components of the mineralization mixture is introduced at a temperature less than a temperature of the formation, the method includes maintaining the solution in the formation such that the solution temperature rises to a temperature of the formation. In such embodiments, the temperature of the formation may be 130° C. or less.

The method of one or more embodiments may include monitoring and analyzing geochemical changes such that CO₂ sequestration is verified. In some embodiments, the method includes monitoring the production of minerals in the formation. Monitoring geochemical changes, the production of minerals, or both may include analyzing core samples from an injection zone of a formation, which can provide information regarding mineralogical transformations downhole. In some embodiments, the method includes geochemical modeling coupled with downhole fluid sampling to assess and determine mineral trapping potential. As one of ordinary skill may appreciate, geochemical modeling may be performed on a computer in electronic communication with one or more on-site units located proximate to a formation of interest.

In some embodiments, the biosequestration of a CO₂ containing fluid can depend on formation temperature, formation pressure, temperature of the CO₂ containing fluid and the EICP solution, or combinations thereof. In a laboratory setting, the biomineralization process can take place rapidly, (e.g., within hours). For example, under controlled conditions in a laboratory experiment, the formation of calcite may take place within one hour using EICP treatment. At larger scales (e.g., on-site applications or in complex geological formations), biomineralization and sequestration of CO₂ may require timeframes ranging from weeks to months.

Embodiments of the present disclosure may provide at least one of the following advantages. The compositions, systems, and methods in accordance with one or more embodiments herein may provide an eco-friendly, cost efficient, and fast acting process for CO₂ sequestration and hardening of formation rock via the deposition of carbonate precipitation. The EICP composition of one or more embodiments may be used in a variety of formations having relatively high formation temperatures. The EICP solution of one or more embodiments is capable of stabilizing the urease enzyme to withstand relatively high formation temperatures (e.g., temperatures of about 70° C. to about 130° C.) and creates nucleation points for carbonate formation as a sequestration agent. The precipitating carbonate from the aqueous solution acts as sequestration agent. The sequestration agent may aid in reinforcing rock strength at high temperature (for example, at least 70° C.) in a formation.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method of biomineralizing CO₂ in a formation, the method comprising:
injecting a CO₂-containing fluid and an EICP solution in the formation to form a mineralization mixture; and
forming a carbonate precipitate in the formation from the mineralization mixture.

2. The method of claim 1, wherein the EICP solution comprises urea, one or more polysaccharides, a casein protein, a protease, an ionic compound, and a urease.

3. The method of claim 2, wherein the one or more polysaccharides are selected from the group consisting of xanthan gum, guar gum, and combinations thereof.

4. The method of claim 2, where the casein protein comprises a micellar casein protein.

5. The method of claim 2, where the ionic compound comprises sodium chloride.

6. The method of claim 1, where the mineralization mixture further comprises a precipitating agent.

7. The method of claim 2, wherein the EICP solution further comprises a metal cation source.

8. The method of claim 7, wherein the metal cation source comprises a calcium ion.

9. The method of claim 7, wherein the metal cation source comprises calcium chloride dihydrate.

10. The method of claim 1, further comprising:
dissolving one or more metal cations present in a rock of the formation in the mineralization mixture.

11. The method of claim 1, wherein forming the carbonate precipitate comprises:
saturating the mineralization mixture with the carbonate precipitate, thereby shifting a reaction equilibrium of the mineralization mixture to form the carbonate precipitate.

12. The method of claim 11, further comprising increasing a pH of the mineralization mixture, wherein increasing the pH of the mineralization mixture comprises forming ammonia in the mineralization mixture.

13. The method of claim 1, wherein the injecting further comprises:
introducing a first solution and a second solution to the formation; and
combining the first solution and the second solution in the formation to form the EICP solution.

14. The method of claim 13, wherein the first solution comprises urea, one or more polysaccharides, casein protein, protease, and an ionic compound, and wherein the second solution comprises a urease.

15. The method of claim 1, wherein the EICP solution is introduced into the formation as one solution.

16. The method of claim 1, further comprising maintaining the formation such that the mineralization mixture achieves a temperature in a range from about 20 to 130° C.

17. The method of claim 1, where one or more components of the mineralization mixture is introduced at a temperature less than a temperature of the formation, and the method further comprises maintaining the solution in the formation such that the solution temperature rises to a temperature in a range from about 70 to 130° C.
